(19) United States
(12) Patent Application Publication
(10) Pub. No.: US 2026/0137219 A1
(43) Pub. Date: May 21, 2026

(54) ADJUSTABLE CRIB MATTRESS SUPPORT

(71) Applicant: Hudson Harbor Labs LLC, Weehawken, NJ (US)

(72) Inventors: Sejal Patel, Weehawken, NJ (US); Deepan Patel, Weehawken, NJ (US); William J. McLeod, Hoboken, NJ (US)

(73) Assignee: Hudson Harbor Labs LLC, Weehawken, NJ (US)

(21) Appl. No.: 19/391,394

(22) Filed: Nov. 17, 2025

Related U.S. Application Data
(60) Provisional application No. 63/722,884, filed on Nov. 20, 2024.

(51) Int. Cl. A47D 9/00; A47C 31/12; A61B 5/00; A61B 5/024

(52) U.S. Cl. CPC A47D 9/012; A47C 31/123; A61B 5/02405; A61B 5/6892; A61B 2503/04

(57) ABSTRACT

A crib includes a walls at least partially defining a crib enclosure, a primary support structure for supporting the walls relative to a surface, a fixation structure fixed relative to the walls, a mattress support translatable vertically relative to the fixation structure and within the crib enclosure, a secondary support structure fixed to the mattress support for supporting the mattress support relative to the surface, and an actuator for vertically translating the mattress support relative to the fixation structure. A mattress support supports a crib mattress within a crib enclosure.

### CROSS-REFERENCE TO RELATED APPLICATION

This application takes priority from U.S. Provisional Patent Application No. 63/722,884 filed November 20, 2024, the contents of which are incorporated by reference herein in their entirety.

### FIELD OF THE INVENTION

The invention relates to an adjustable height mattress support for use in a crib and to a crib with an adjustable height mattress support.

### BACKGROUND

Height adjustable cribs are well known, and generally provide for adjusting a mattress height within a crib. Such height adjustments are generally provided because a different height may be appropriate for children of different ages and sizes.

Such height adjustments are generally performed by disassembling a crib and reassembling it in a different configuration. The intent for such a design is for a mattress height to be adjusted as a child ages, but not on a regular basis. Accordingly, a convertible crib may provide several different configurations, including different mattress height settings and a bed configuration wherein a wall may be removed allowing a crib to be used for a child after they age or size out of the different configurations.

When an infant or child requires attention or care, the child often must be removed from or adjusted within a crib. Accordingly, when the crib mattress is at a low height, an attendant caring for the child typically must bend over to retrieve the child. When the mattress is at a standard setting (including the higher setting of a typical adjustable crib), such frequent bending and lifting may lead to back pain. This is particularly true where a crib wall height requires an attendant to maintain poor posture while lifting the child.

While cribs have in the past addressed this problem by using “drop-side” configurations, where a side of a crib lowers to allow for easier access, such a configuration is generally considered unsafe and is no longer used.

While a one time, or infrequent, adjustment may allow for a child to continue using a crib as they age and grow, such adjustments to not alleviate pain caused by frequent bending over to retrieve a child. In fact, lowering a crib mattress height for a larger and heavier child exacerbates such pain, as a heavier child will require bending over further for lifting.

Further, many cribs provide fixed legs in order to provide a stable foundation, while others provide wheels or casters fixed at the base of the legs in order to allow for easy movement of the crib. However, parents and attendants may prefer a crib that is easily movable at some times and stably fixed in a location at other times. Indeed, an attendant who frequently removes a child from a crib may wish the crib to be sturdy and unmoving when placing the child in or removing the child from the crib, but may wish to be able to easily move the crib once the child is placed within the crib.

Cribs with wheels or casters often provide locking mechanisms. However, such locking mechanism are not always reliable, and even when properly applied, a crib resting on such wheels or casters is not as sturdy as a crib resting on traditional legs.

Accordingly, there is a need for a crib that has a mattress pad that is easily adjustable during regular use rather than only by way of disassembly. There is a further need for such a crib in which a user may be able to switch between a sturdy foundation and a wheel or caster based foundation as needed.

There is a further need for an adjustable height mattress support that can provide at least some of these features to existing cribs.

### SUMMARY

Various implementations of adjustable crib mattress supports are described in this disclosure. In some versions, a crib is provided that includes such an adjustable mattress support. In other versions, a portable crib mattress support is provided that can be paired with a variety of existing cribs.

In some embodiments, a crib includes a plurality of walls at least partially defining a crib enclosure, a primary support structure for supporting the plurality of walls relative to a surface, a fixation structure fixed relative to the plurality of walls, a mattress support translatable vertically relative to the fixation structure and within the crib enclosure, a secondary support structure fixed to the mattress support for supporting the mattress support relative to the surface, and an actuator for vertically translating the mattress support relative to the fixation structure.

In some embodiments, the crib has a first configuration wherein the crib and mattress support are supported relative to the surface by the primary support structure and not the secondary support structure and a second configuration wherein the crib and mattress support are supported relative to the surface by the secondary support structure and not the primary support structure.

In some such embodiments, the crib has a third configuration in which both the primary support structure and the secondary support structure are simultaneously in contact with the surface.

In some embodiments, a height of the mattress support is continuously adjustable within its range of motion, such that the mattress support can be set to a plurality of heights within the first configuration.

In some embodiments, the secondary support structure includes wheels or casters and, when in the second configuration, the crib and mattress support are supported relative to the surface by the wheels or casters such that in the second configuration, the crib is mobile.

In some embodiments, the actuator is or includes at least one of a scissor jack, a screw jack, a linear actuator, a motorized actuator, and a manually operated mechanical actuator. In some such embodiments, the actuator is configured to set the mattress support to one of a plurality of predetermined heights.

In some embodiments, the crib includes a sensor for determining if an obstruction is in a path of the mattress support, and a controller is configured to prevent movement of the mattress support upon detection of an obstruction by the sensor.

In some embodiments, the crib includes a weight sensor for determining a weight of an object placed on the mattress support, and the mattress support has a primary maximum weight at which actuation of the actuator is safe, and a determination that the weight of the object is greater than the primary maximum weight prevents movement of the actuator. In some such embodiments, the mattress support has a secondary maximum weight at which use of the mattress support is safe, wherein the secondary maximum weight is higher than the primary maximum weight.

In some embodiments, the crib includes a sound generator for generating white noise, where the sound generator obscures any noise generated by a motor associated with the actuator.

In some embodiments, the crib includes a sensor for detecting heart rate variability data associated with a child located within the crib.

In some embodiments, the primary support structure includes a first plurality of legs for supporting the plurality of walls, and the secondary support structure includes a second plurality of legs fixed to the mattress support.

In some embodiments, the fixation structure is a platform or shelf fixed to at least one of the plurality of walls and to which the actuator is mounted.

In some embodiments, the the fixation structure is a mounting structure or location integrated into at least one of the plurality of walls for mounting the actuator.

Also provided is a mattress support for use in a crib. Such a mattress support includes a mattress platform sized for supporting a crib mattress within a crib enclosure, a primary support structure for supporting the mattress platform relative to a surface, and an actuator for vertically translating the mattress platform relative to a foot of the primary support structure.

In some embodiments, the actuator is located within the primary support structure, such that the primary support structure expands or contracts in order to adjust a height of the mattress platform. In some such embodiments, the mattress support includes a primary support structure housing for fully enclosing the actuator.

In some embodiments, the mattress platform includes a plurality of independent panels, and a footprint of the primary support structure corresponds substantially to a size of each of the plurality of independent panels.

Also provided is a crib including a plurality of walls at least partially defining a crib enclosure, a first support structure for supporting the plurality of walls relative to a surface, a mattress platform translatable vertically relative to the plurality of walls and within the crib enclosure, a second support structure fixed to the mattress platform for supporting the mattress support relative to the surface, and an actuator for vertically translating the mattress platform relative to the second support.

### BRIEF DESCRIPTION OF THE DRAWINGS

FIG. 1 is a top perspective view of a crib in a first configuration in accordance with this disclosure.

FIG. 2 is a bottom perspective view of the crib of FIG. 1 in the first configuration.

FIG. 3 is a front view of the crib of FIG. 1.

FIG. 4 is a side view of the crib of FIG. 1.

FIG. 5 is sectioned view of the crib of FIG. 1.

FIG. 6 is a front view of the crib of FIG. 1 in a second configuration.

FIG. 7 is a top perspective view of a crib in accordance with this disclosure.

FIG. 8 is a bottom perspective view of the crib of FIG. 7.

FIG. 9 is a front view of the crib of FIG. 7.

FIG. 10 is a side view of the crib of FIG. 7.

FIG. 11 is sectioned view of the crib of FIG. 7.

FIG. 12 is a top perspective view of an adjustable height mattress support in the context of a crib in accordance with this disclosure.

FIG. 13 is a bottom perspective view of the mattress support of FIG. 12.

FIG. 14 is a front view of the mattress support of FIG. 12.

FIG. 15 is a side view of the mattress support of FIG. 12.

FIG. 16 is sectioned view of the mattress support of FIG. 12.

FIG. 17 is an exploded view of the mattress support of FIG. 12.

FIG. 18 shows the mattress support of FIG. 12 in a first configuration.

FIG. 19 shows the mattress support of FIG. 12 in a second figuration.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

Various embodiments of an adjustable height crib are shown and described. In some embodiments, the crib has the same basic structure of a traditional crib. However, in some embodiments, a mattress platform is not fixed to the main legs of the crib, and is instead fixed to a movable and vertically translatable platform. Such a platform may be moved by a powered or unpowered actuator, such as a screw jack which is in turn attached to walls or legs of the crib. The lifting system may detect a maximum safe height using sensors, such as a hall effect sensor, and it can automatically detect a lowest safe crib position, also by way of the hall effect sensor. Other approaches are contemplated as well.

In a lower position, the embodiment may put attached caters in contact with the floor and lift the main crib frame a small distance in order to allow for wheeled modes and stable modes.

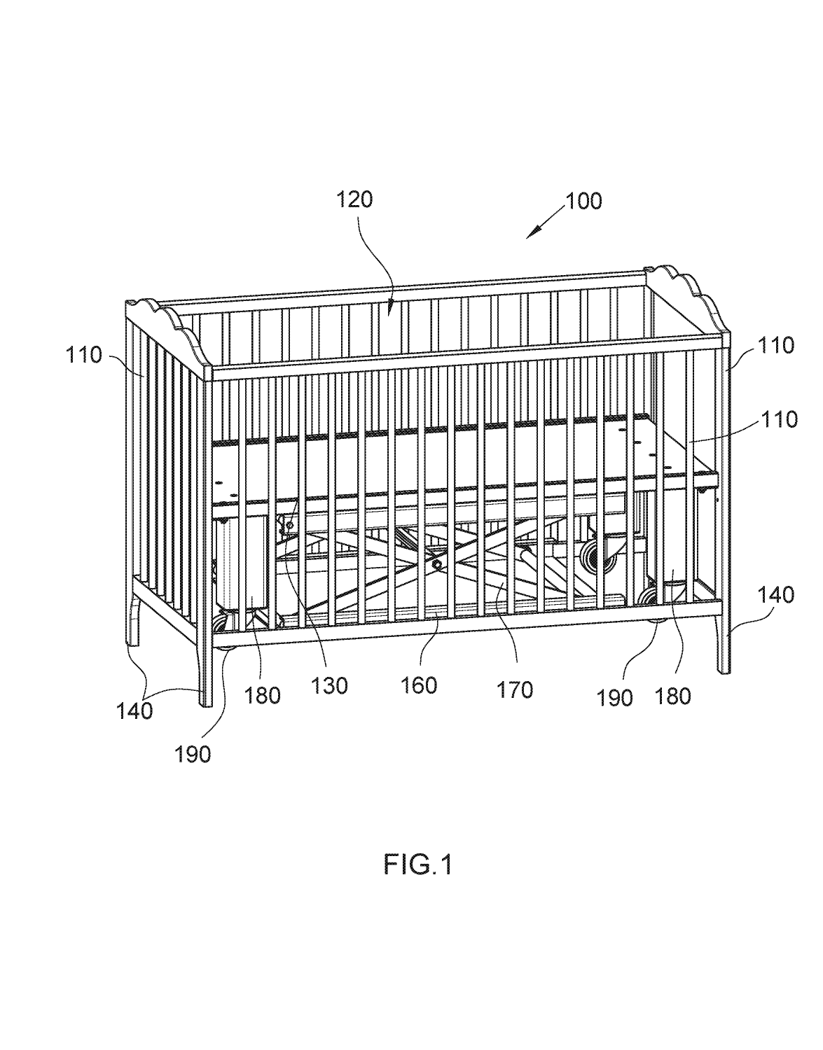

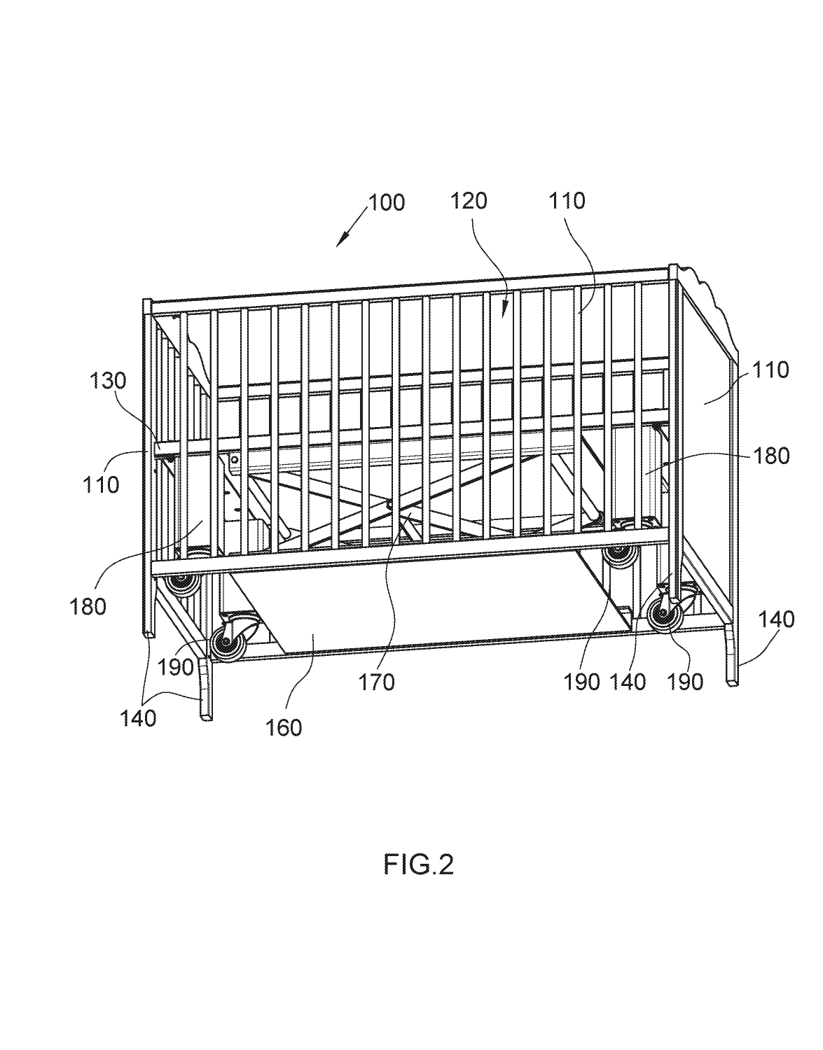

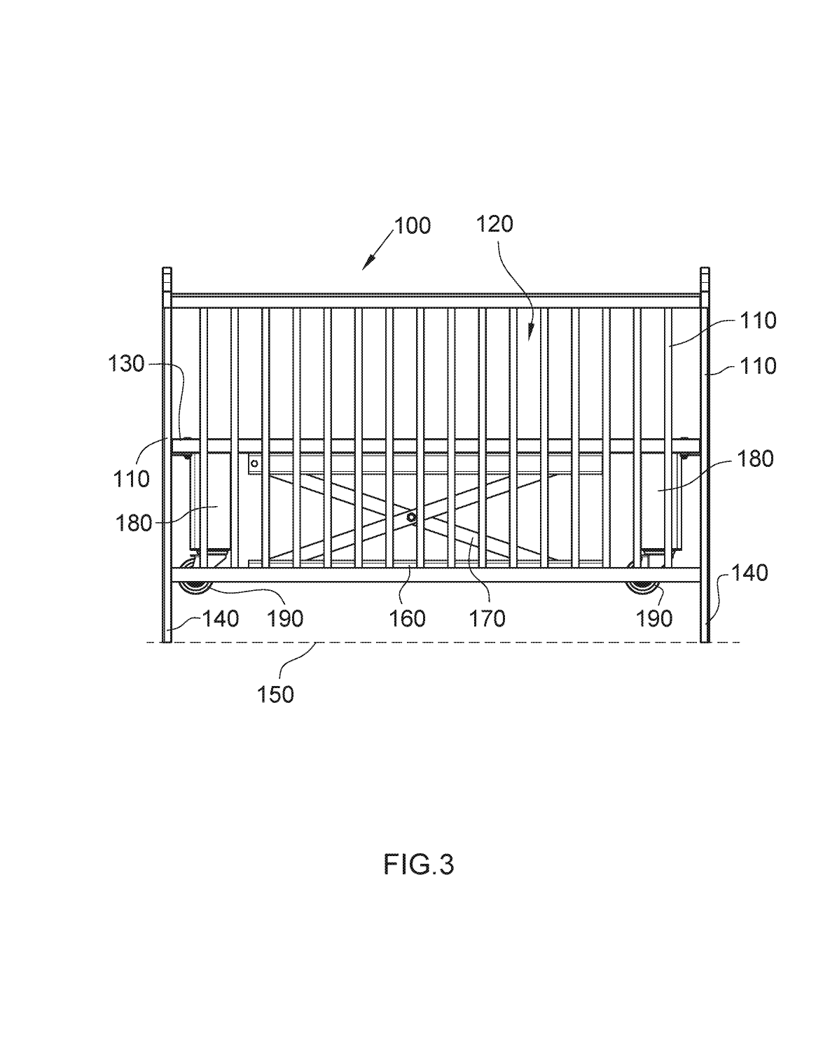

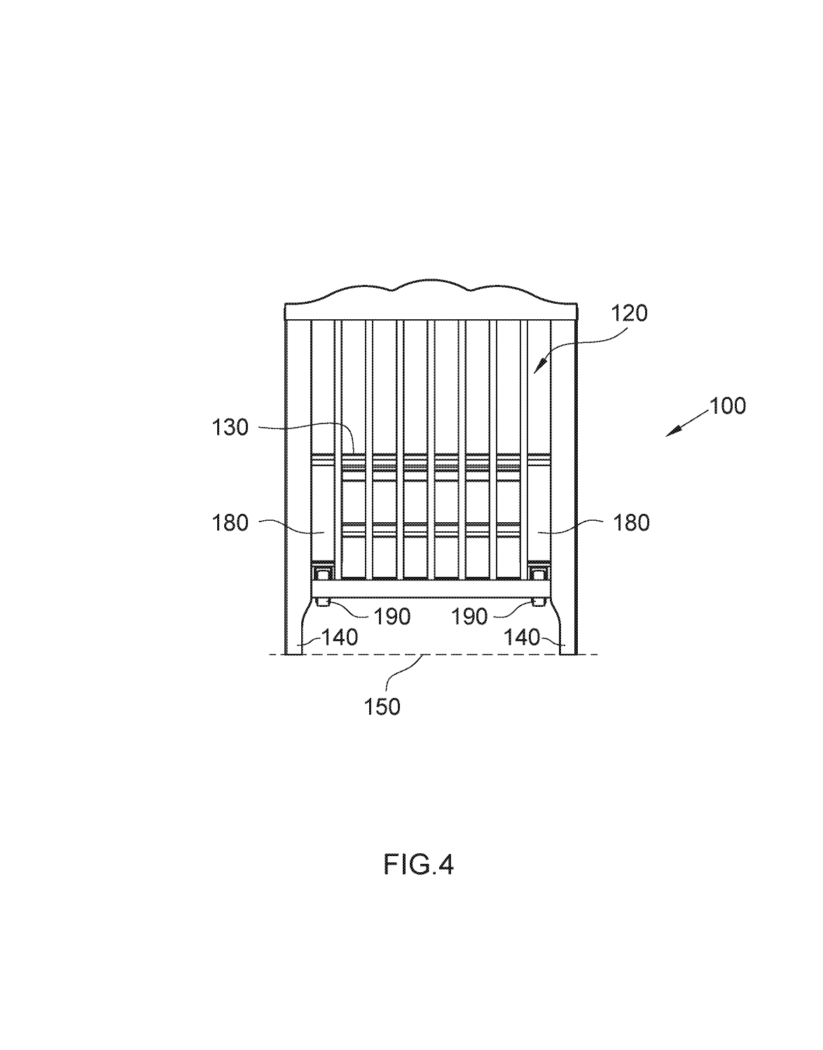

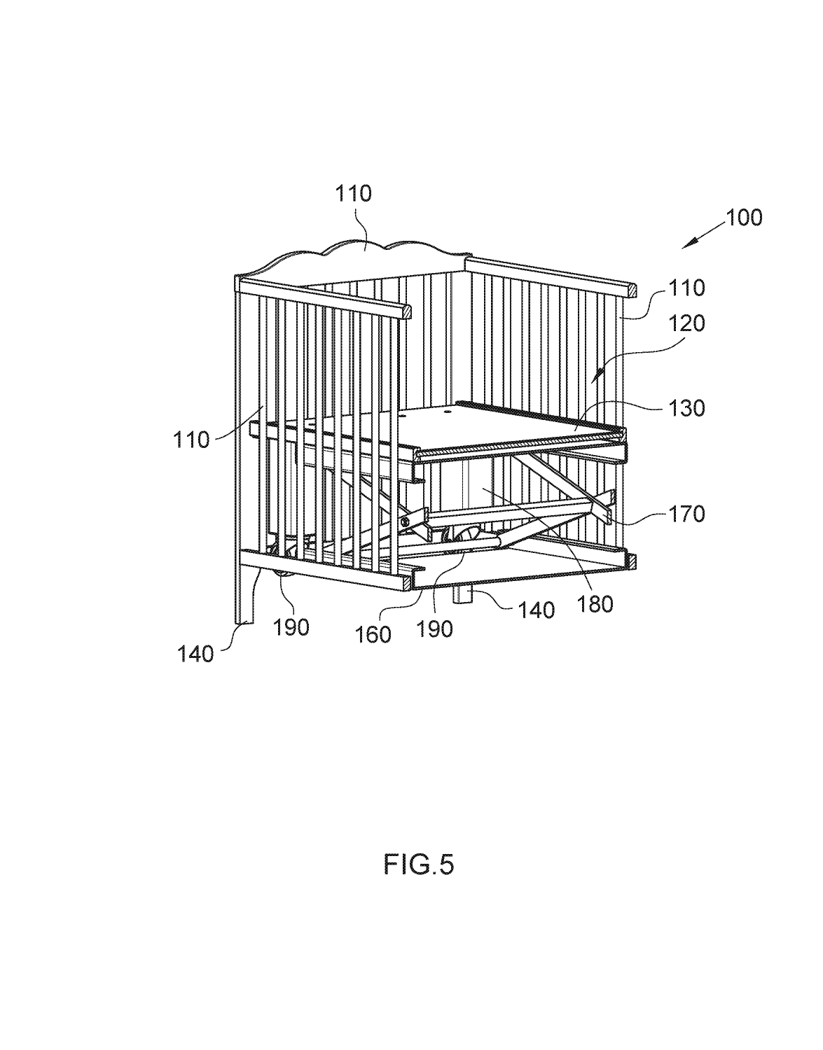

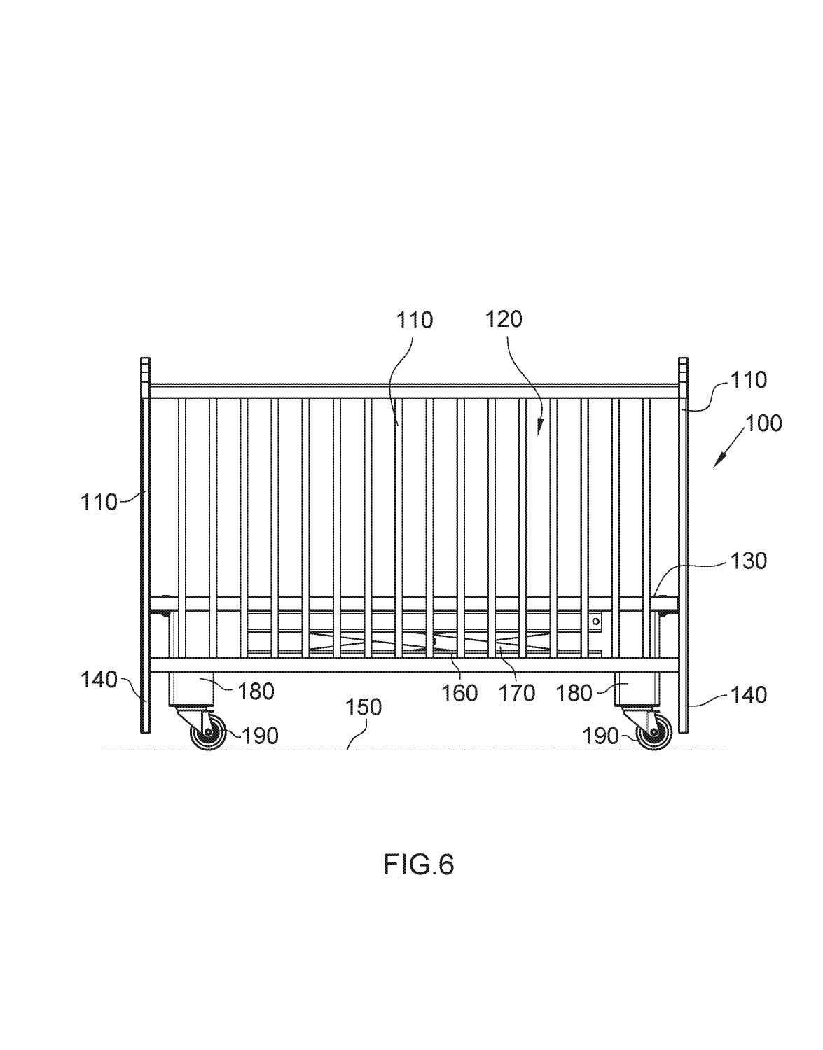

FIG. 1 is a top perspective view of a crib 100 in accordance with this disclosure. FIG. 2 is a bottom perspective view of the crib 100 of FIG. 1 in a first configuration. FIG. 3 is a front view of the crib 100 of FIG. 1. FIG. 4 is a side view of the crib 100 of FIG. 1. FIG. 5 is sectioned view of the crib 100 of FIG. 1. FIG. 6 is a front view of the crib 100 of FIG. 1 in a second configuration.

As shown, the crib 100 includes a plurality of walls 110 that define an interior space, or crib enclosure 120. Typically, the crib provides four walls 110 defining a rectangular shape as shown, but other configurations are contemplated as well. Such walls 110 may be panels, or they may be defined by slats or bars.

A mattress support 130 is then provided within the crib enclosure 120, the mattress support typically supporting a crib mattress (not shown) that an infant or child would be placed on.

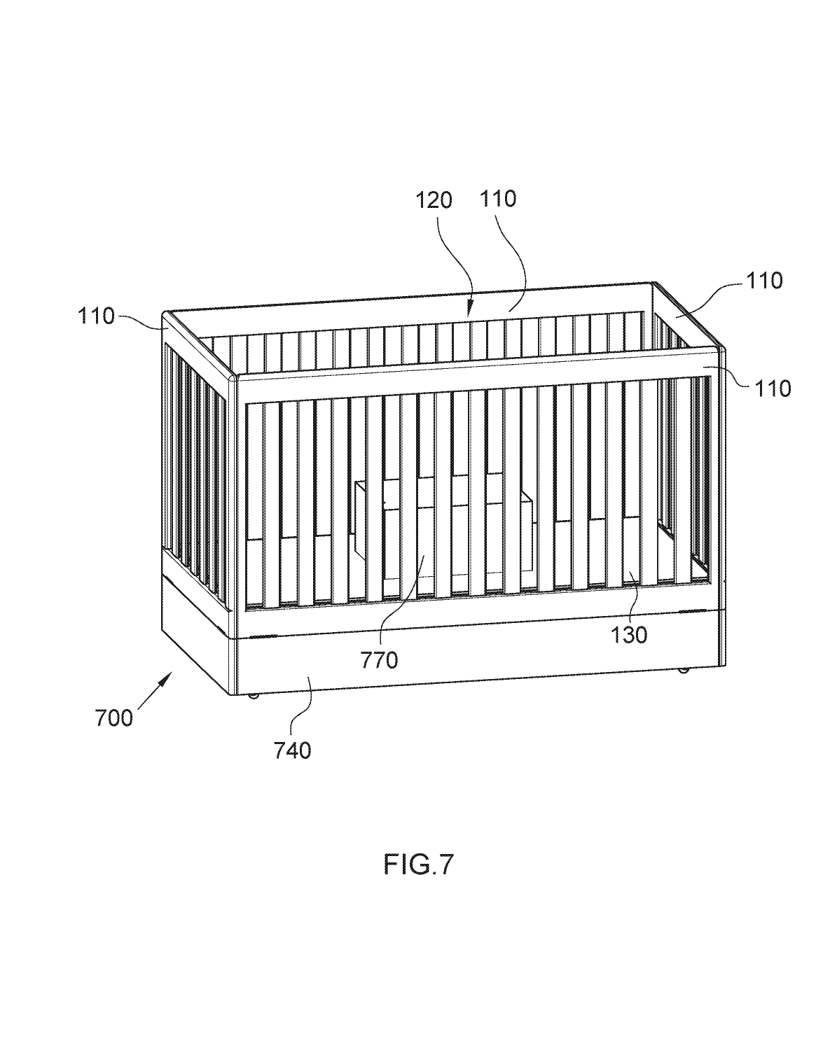

The crib 100 further includes a primary support structure 140 for supporting the plurality of walls 110 relative to a surface 150. The surface 150 may be a floor, and the primary support structure 140 may be legs, or it may be a different foundation structure, such as that shown in FIG. 7. In any event, such a primary support structure 140 locates the crib 100 relative to the surface 150.

The crib 100 further includes a fixation structure 160 fixed relative to the plurality of walls 110. Such a fixation structure 160 serves as an internal foundation for supporting the mattress support 130. The mattress support 130 is then translatable vertically relative to the fixation structure 160 and within the crib enclosure 120.

Such a fixation structure 160 may be, for example, a platform or shelf, such as that shown, and such a platform or shelf may serve as a foundation for supporting an actuator 170 for vertically translating the mattress support 130 relative to the fixation structure. Alternatively, the fixation structure 160 may be a mounting structure or location integrated into at least one of the walls 110, which can then serve as a mounting location or structure for supporting the actuator 170.

In the embodiment shown, the fixation structure 160 is a platform which supports a scissor jack as an actuator 170. However, many different types of actuators are contemplated, including pistons, linear actuators, screw jacks, inflatable actuators, and others.

The crib 100 further includes a secondary support structure 180 fixed to the mattress support 130 for supporting the mattress support relative to the surface 150. Such a secondary support structure 180 may be legs, and it may include wheels or casters 190, as shown. The primary support structure 140 and the secondary support structure 180 may rest on the surface 150 simultaneously or interchangeably, such that the entire crib 100, including the walls 110 may rest on either one of or both of the primary support structure and the secondary support structure. This may depend on a configuration in which the crib 100 is placed.

The crib 100 then typically has at least two configurations. In the first configuration, shown in FIG. 3, for example, the crib 100 and mattress support 130 are supported relative to the surface 150 by the primary support structure 140 and not the secondary support structure 180. In the embodiment shown, the crib 100 is then supported by the legs 140 and the walls 110 are directly supported by the associated support structure. Accordingly, when the mattress support 130 is raised vertically, the secondary support structure 180 fixed to the mattress support is typically raised as well, and would therefore be above a level associated with the primary support structure 140.

In a second configuration, shown in FIG. 6, the crib 100 and mattress support 130 are supported relative to the surface 150 by the secondary support structure 180 and not the primary support structure 140. Accordingly, when the mattress support 130 is lowered vertically, the secondary support structure 180 is typically lowered as well, and would therefore be below a level associated with the primary support structure 140.

As such, when the mattress support 130 is raised to a higher position by way of the actuator 170 relative to the fixation structure 160, the secondary support structure 180 may be lifted off the surface 150 if it had previously been in contact with that surface, allowing the crib 100 to be fully supported by the primary support structure 140. Similarly, when the mattress support 130 is lowered to a lower position by way of the actuator 170 relative to the fixation structure 160, the secondary support structure 180 may be lowered to the surface 150 and the primary support structure 140 may then be lifted off the surface, such that the crib 100 is fully supported by the secondary support structure.

As noted above, the secondary support structure 180 may terminate in wheels or casters 190. Accordingly, when in the second configuration, the crib 100 and mattress support 130 may be supported relative to the surface 150 by the wheels or casters 190 such that in the second configuration, the crib is mobile. Such wheels or casters 190 may be lockable, such that even in the second configuration, the crib 100 could be locked in place.

In some embodiments, the crib 100 may be provided with a third configuration, not shown, in which both the primary support structure 140 and the secondary support structure 180 are simultaneously in contact with the surface 150. Such a configuration may provide for additional stability, and would typically correspond to the lowest mattress support 130 position at which the crib 100 is not raised off of its primary support structure 140. Where the secondary support structure 180 is provided with wheels or casters 190, such a third configuration represents the lowest level for the mattress support 130 at which the crib continues to be supported by, e.g., fixed legs 140.

In some embodiments, the height of the mattress support 130 is continuously adjustable within its range of motion. Accordingly, the mattress support 130 may be set to a plurality of heights within the first configuration. Alternatively, in some embodiments, the mattress support 130 is not continuously adjustable, but still allows for selection of specific heights from a plurality of predetermined heights. Such heights may be based on a configuration associated with the actuator 170, or they may be defined by, e.g., detents in one or more components of a mechanical system.

Accordingly, the actuator 170 may be a motorized actuator, and the motorized actuator may be configured to set the mattress support 130 to one of a plurality of predetermined heights.

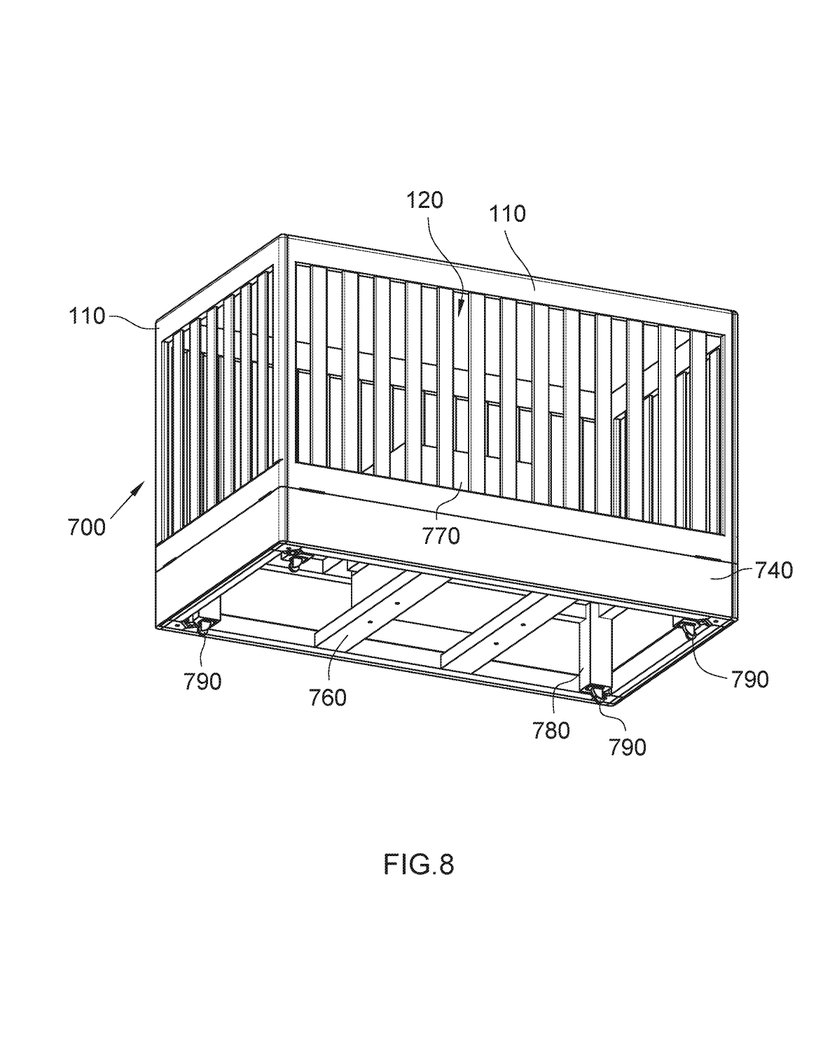

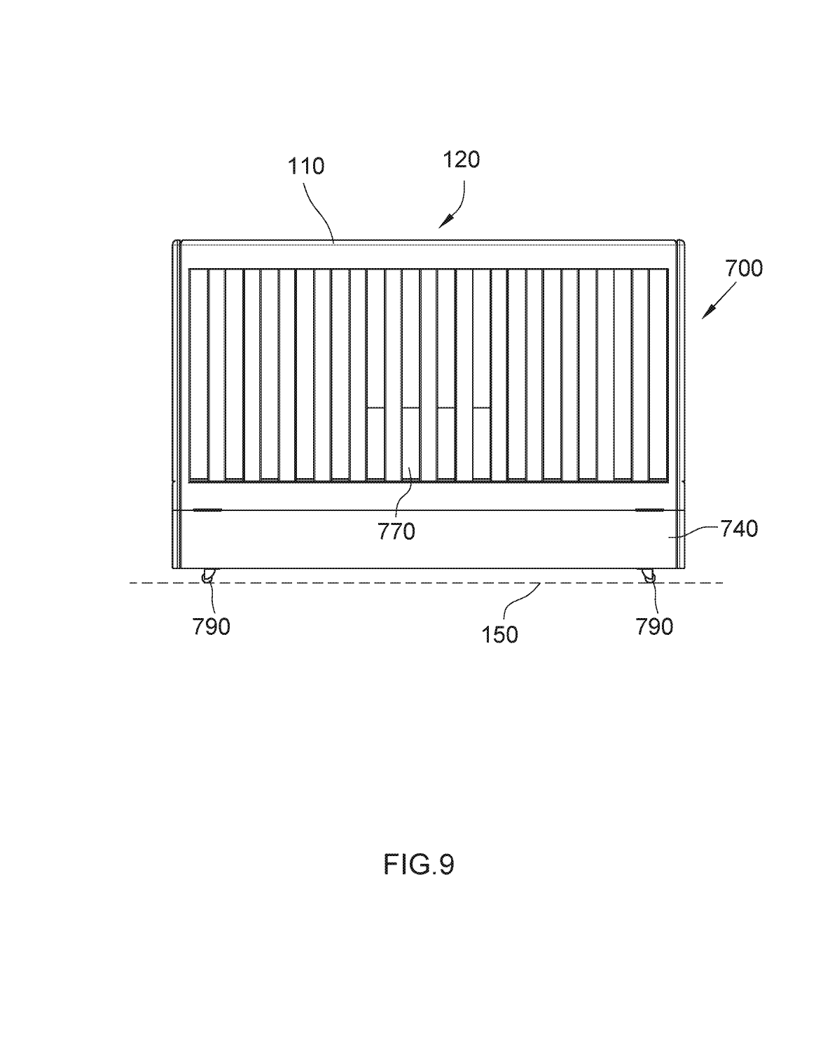

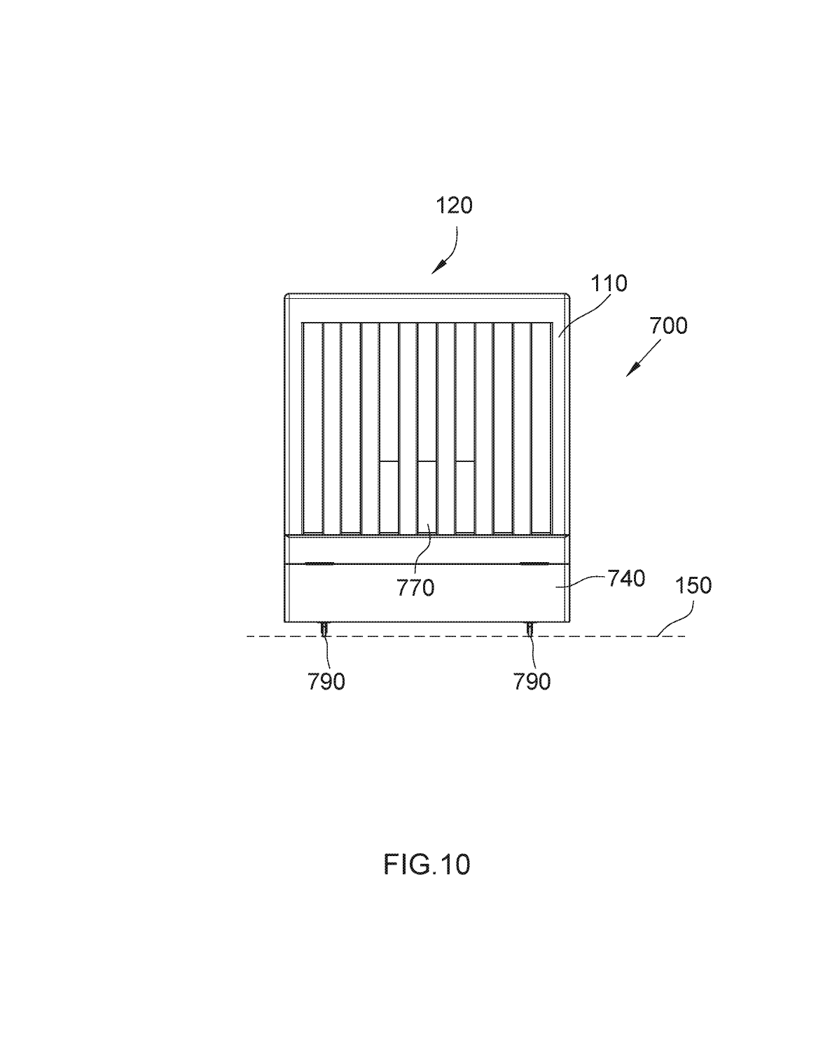

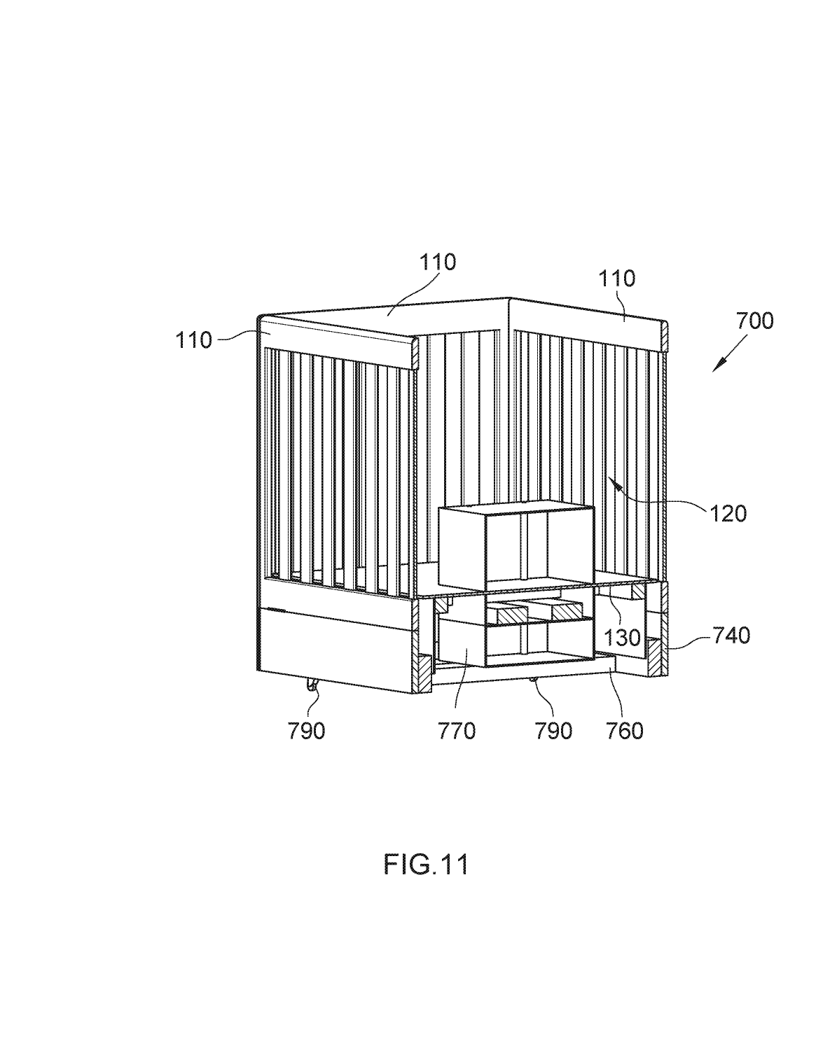

FIG. 7 is a top perspective view of a crib 700 in accordance with this disclosure. FIG. 8 is a bottom perspective view of the crib 700 of FIG. 7. FIG. 9 is a front view 700 of the crib of FIG. 7. FIG. 10 is a side view of the crib 700 of FIG. 7. FIG. 11 is sectioned view of the crib 700 of FIG. 7.

The embodiment of the crib 700 of FIG. 7 is generally similar to that discussed above with respect to FIG. 1, and like components are labeled with like reference numerals. Accordingly, the crib 700 includes a plurality of walls 110 that define an interior space, or crib enclosure 120.

A mattress support 130 is then provided within the crib enclosure 120, the mattress support typically supporting a crib mattress (not shown) that an infant or child would be placed on. It is noted that in FIGS. 7-11, a schematic box is shown corresponding to the actuator 770 used in the corresponding embodiment. However, the mattress support 130 is flat, and the top of the schematic box 770 merely represents the highest position achievable for the mattress support 130.

The crib 700 further includes a primary support structure 740 for supporting the plurality of walls 110 relative to a surface 150. The surface 150 may be a floor, and the primary support structure 740 may be a foundation structure fully circumscribing the crib 700. Such an approach may allow the mechanisms described herein to be hidden during normal usage of the crib 700. In any event, such a primary support structure 740 locates the crib 700 relative to the surface 150.

The crib 100 further includes a fixation structure 760 fixed relative to the plurality of walls 110. Such a fixation structure 760 serves as an internal foundation for supporting the mattress support 130. The mattress support 130 is then translatable vertically relative to the fixation structure 760 and within the crib enclosure 120.

Such a fixation structure 760 may be crossbeams, such as those shown, and such crossbeams may serve as a foundation for supporting the actuator 770 for vertically translating the mattress support 130 relative to the fixation structure. The use of such crossbeams 760 may allow the actuator 770 to be located centrally within the crib 700.

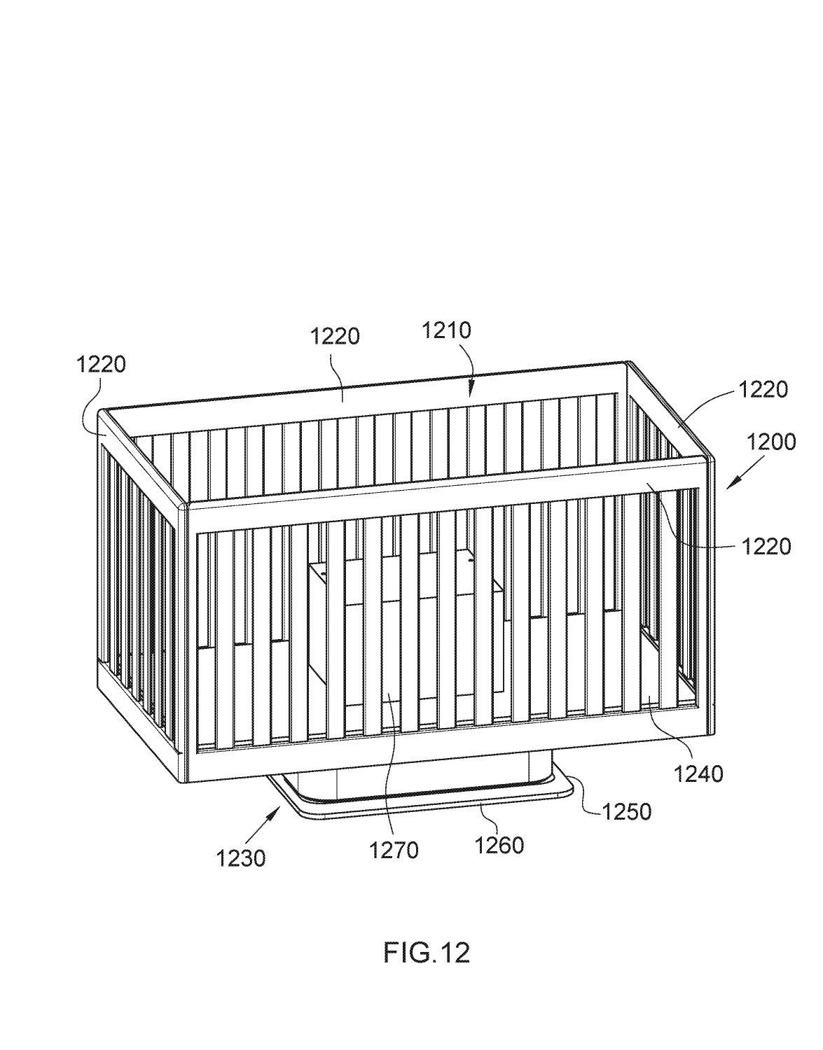

In the embodiment shown, the fixation structure 760 is crossbeams supporting a schematic actuator 770. As noted above, many different types of actuators are contemplated, including scissor jacks, pistons, linear actuators, screw jacks, inflatable actuators, and others. In the embodiment shown, a scissor jack may be located within a sealed housing 770 associated with the actuator thereby enhancing safety associated with the crib. Such a sealed housing is described in more detail below with respect to the embodiment of FIG. 12.

The crib 700 further includes a secondary support structure 780 fixed to the mattress support 130 for supporting the mattress support relative to the surface 150. Such a secondary support structure 780 may be legs, and it may include wheels or casters 790, as shown. The primary support structure 740 and the secondary support structure 780 may rest on the surface 150 simultaneously or interchangeably, such that the entire crib 700, including the walls 110 may rest on either one of or both of the primary support structure and the secondary support structure. This may depend on a configuration in which the crib 700 is placed.

The various configurations of the crib 700 of FIG. 7 function similarly to those described above with respect to the crib 100 of FIGS. 3 and 6.

Additional features may be provided in the context of either the crib 100 of FIG. 1 or the crib 700 of FIG. 7. Accordingly, in some embodiments, a sensor may be provided for determining if an obstruction in a path of the mattress support 130 while it is being raised or lower. Accordingly, a controller may be configured to prevent movement of the mattress support upon detection of an obstruction by the sensor. Such a sensor may be, e.g., a photoelectric sensor. Accordingly, the crib may have a sensor or a series of safety sensors that use IR beams to detect when an obstruction has crossed the path of the crib.

In some embodiments, a weight sensor may be provided for determining a weight of an object, such as a child, placed on the mattress support 130. In some such embodiments, the crib 100, 700 may have a primary maximum weight at which actuation of the actuator 170, 770 is safe. Accordingly, a determination that the weight of the object is greater than the primary maximum weight prevents movement of the actuator.

In some embodiments, the mattress support 130 may also have a secondary maximum weight at which use of the mattress support is safe. Accordingly, within a certain weight range, use of the crib 100, 700 with the mattress support 130 may be safe, but actuation of the actuator 170, 770 with the child in the crib enclosure 120 may not be safe. Accordingly, the secondary maximum weight may be higher than the primary maximum weight.

Further, the weight sensor may function as a scale, such that the crib 100, 700 may be used to track the weight of a child.

In some embodiments, the crib 100, 700 may generate a warning if the secondary maximum weight is exceeded. Accordingly, if a child is between the primary and secondary maximum weights, the actuator 170, 770 may not function when the child is present, but the crib 100, 700 would not generate a warning.

In some embodiments, the crib may further provide a sound generator for generating, e.g., white noise. Such white noise, or other soothing sounds, may be further utilized to obscure any noise generated by a motor associated with the actuator 170, 770. Accordingly, such a white noise generator may aid in sleeping and mask any motor noise.

In some embodiments, the crib 100, 700 and the corresponding mattress support 130 may further include a sensor for detecting heart rate variability data associate with a child located within the crib. Such data may be utilized to monitor the child’s sleep and ensure their safety. Further, such a sensor may be used to monitor sleep cycles and predict wake times. The sensors may be further used in conjunction with a baby monitor camera, which is in turn linked to a machine vision model for detecting motion versus sleeping babies for training the heart rate variability sensor data.

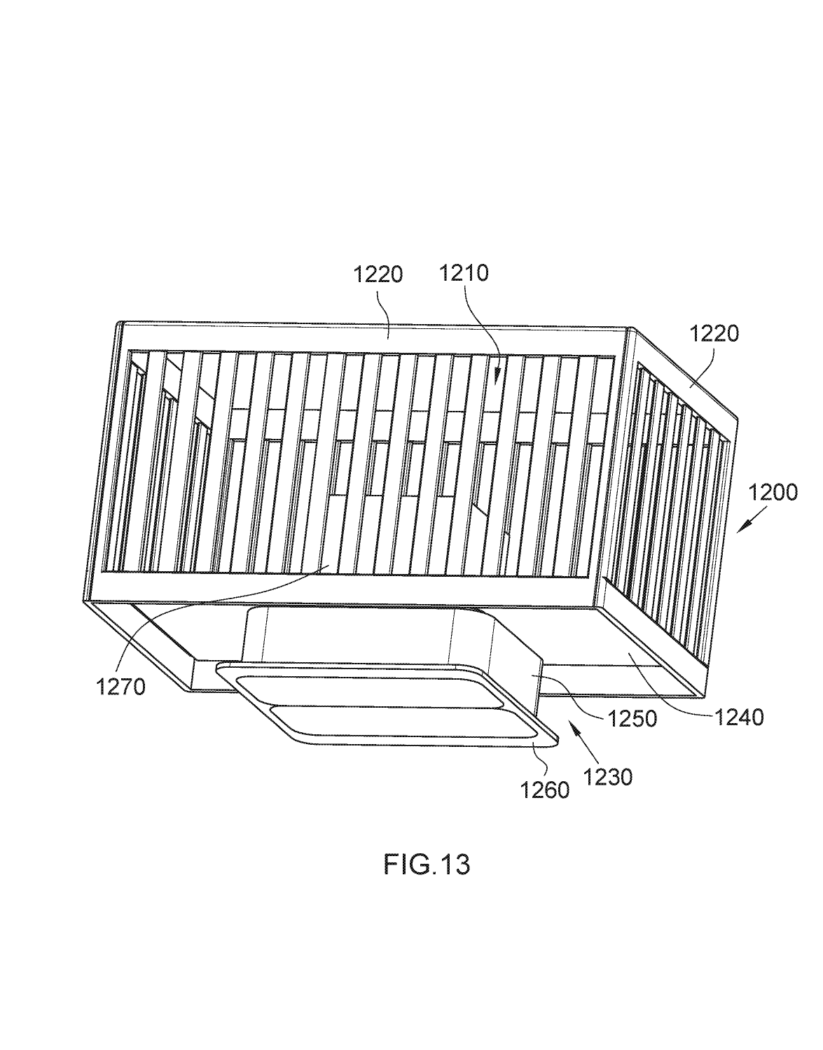

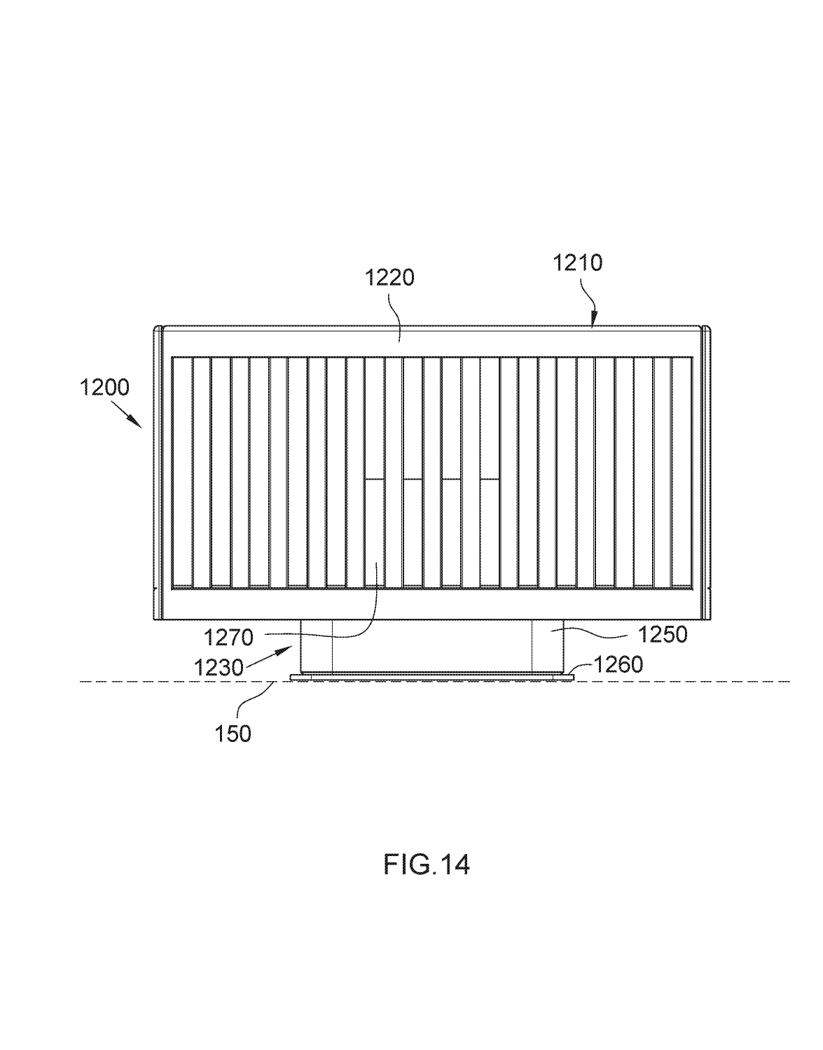

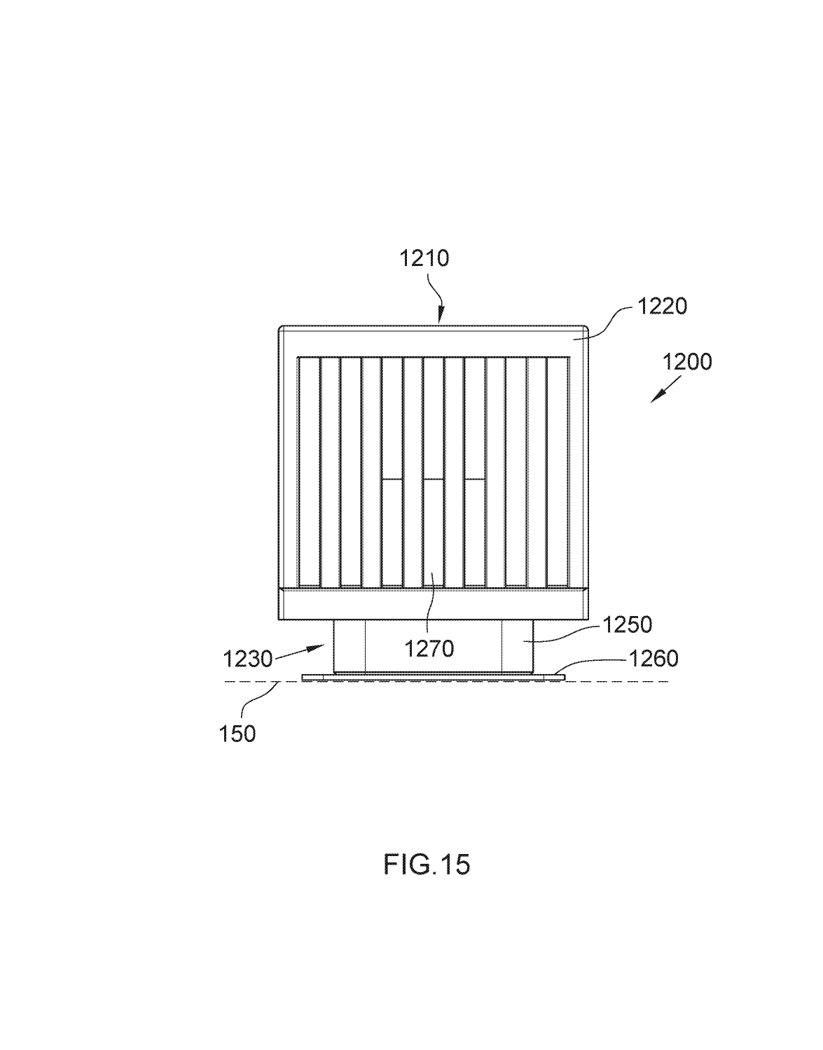

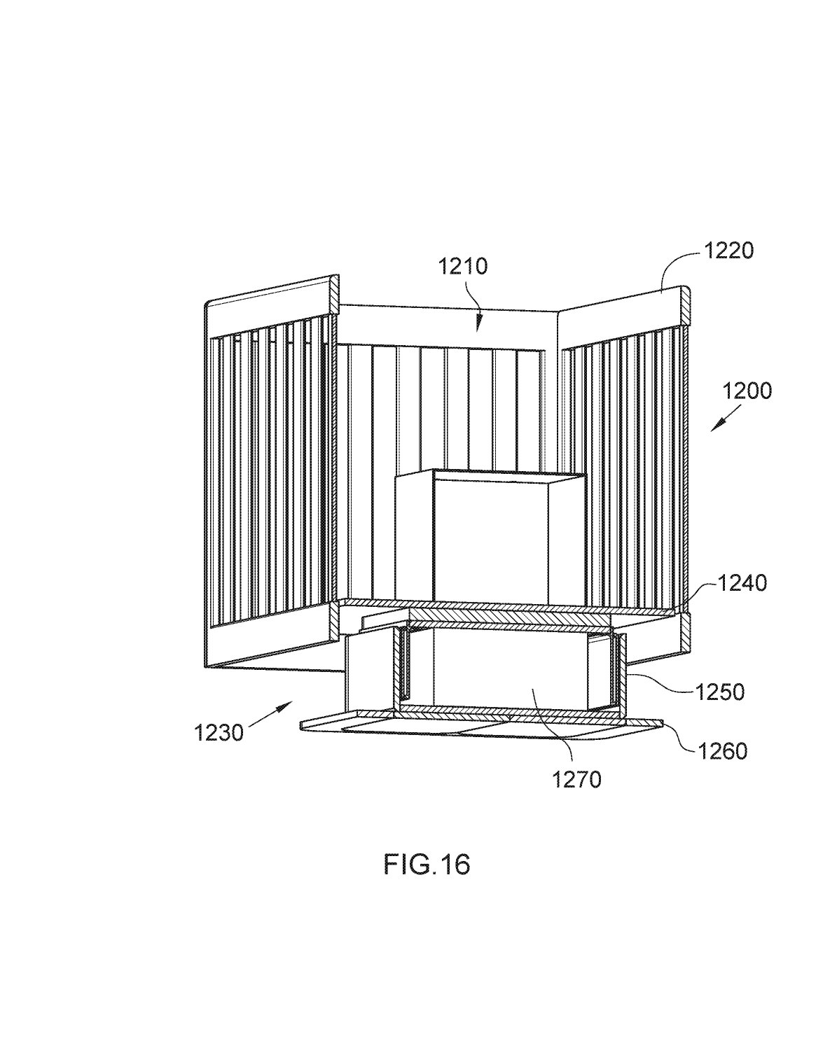

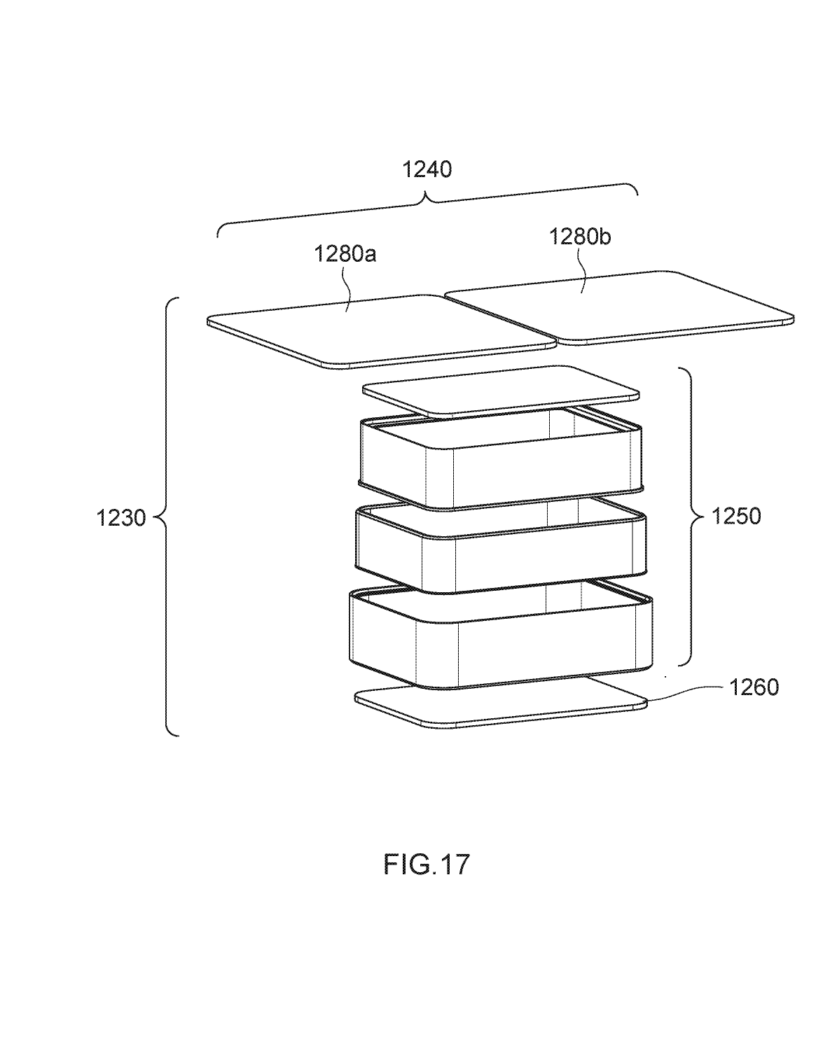

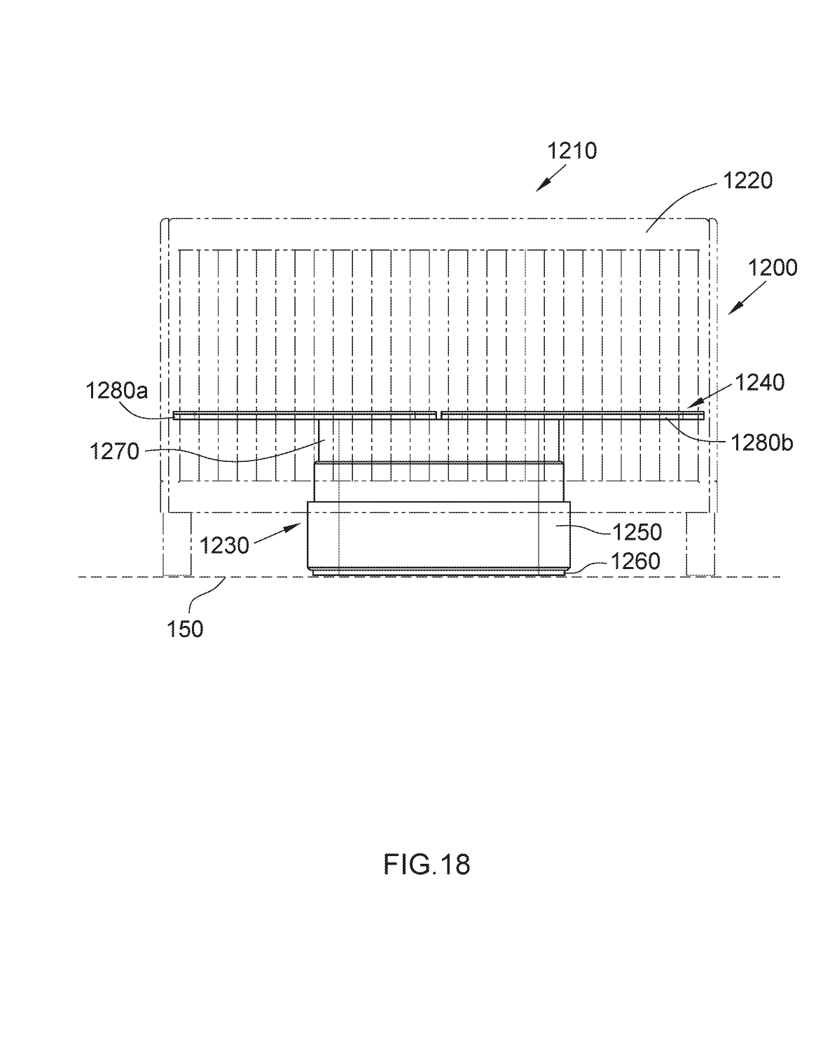

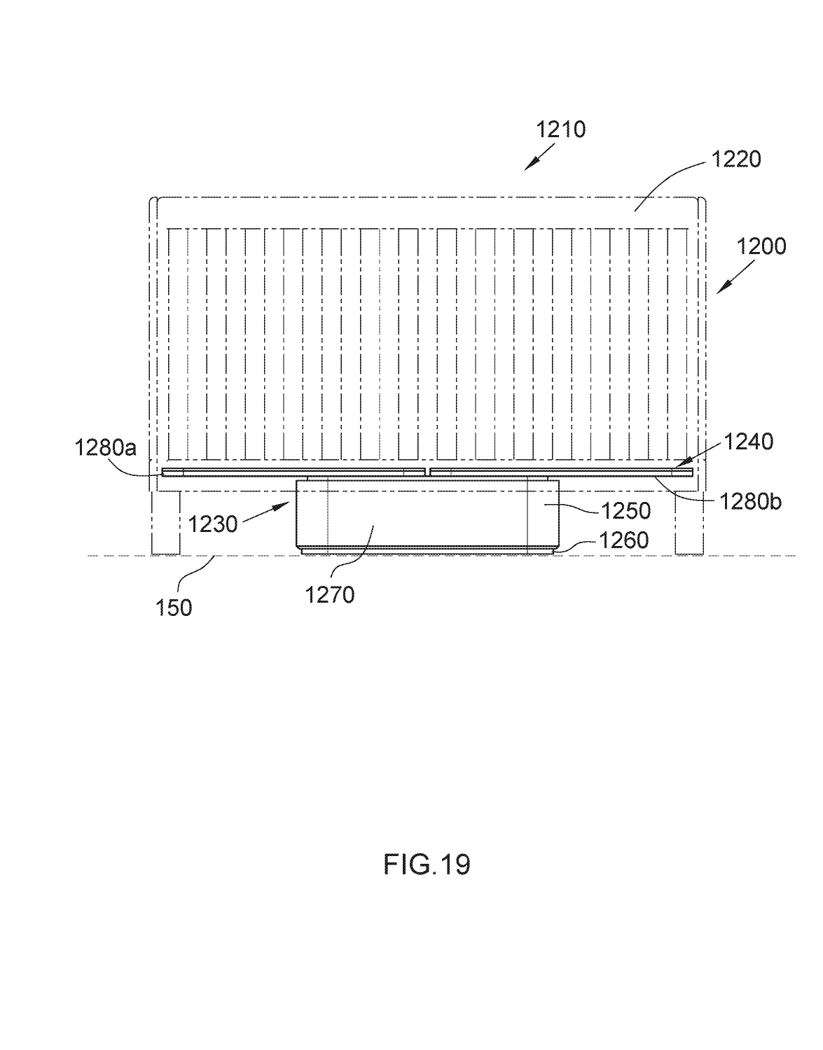

FIG. 12 is a top perspective view of an adjustable height mattress support 1230 in the context of a crib 1200 in accordance with this disclosure. FIG. 13 is a bottom perspective view of the mattress support 1230 of FIG. 12. FIG. 14 is a front view of the mattress support 1230 of FIG. 12. FIG. 15 is a side view of the mattress support 1230 of FIG. 12. FIG. 16 is sectioned view of the mattress support 1230 of FIG. 12. FIG. 17 is an exploded view of the mattress support 1230 of FIG. 12. FIG. 18 shows the mattress support 1230 of FIG. 12 in a first configuration. FIG. 19 shows the mattress support 1230 of FIG. 12 in a second figuration.

As shown, the mattress support 1230 may be provided for use with a crib 1200, but it may not be directly fixed to the crib. Accordingly, the embodiment shown may function as a retrofit to an existing crib in which a traditional mattress support has been removed.

Accordingly, the mattress support 1230 may include a mattress platform 1240 sized for supporting a crib mattress (not shown) within a crib enclosure 1210. Such a crib enclosure 1210 would typically be defined by walls 1220 of the crib 1200. The mattress support 1230 is then provided with a primary support structure 1250 for supporting the mattress platform 1240 relative to a surface 150 and an actuator 1270 for vertically translating the mattress platform 1240 relative to a foot 1260 of the primary support structure 1250.

As shown, the actuator 1270 may be located within the primary support structure 1250 such that a housing for the primary support structure expands or contracts in order to adjust a height of the mattress platform 1230. Accordingly, a portion of the primary support structure 1250, or a housing for the primary support structure, may function as a housing for the actuator 1270. In this discussion, the actuator 1270 and the housing are considered interchangeably, as the actuator may take a variety of forms, so long as it functions to expand within the housing. For example, a scissor lift may be enclosed within the housing 1270 to define the actuator.

Accordingly, a primary support structure housing 1270 may fully enclose the actuator, and as such, may be considered to be a housing for the corresponding actuator. For example, in the embodiment shown, the housing 1270 for the primary support structure 150 may take the form of a telescoping cowling.

In the embodiment shown, the mattress platform 1240 comprises a plurality of independent panels 1280a, b. In the embodiment shown, two such panels are provided. A footprint of the primary support structure 1250 corresponding to the foot 1260 of the same may correspond substantially to a size of each of the plurality of independent panels 1280a, b. Accordingly, the mattress platform 1240 may be shipped in or stored in packaging half the size of a traditional crib mattress, allowing for portability. When assembled and in use, the mattress platform 1240 would typically be sized to correspond to the crib enclosure 1210 as defined by the walls 1220.

As shown in FIGS. 18 and 19, the mattress support 1230 typically has at least two configurations. In the first configuration, shown in FIG. 18, the mattress platform 1240 is supported relative to the surface 150 by the primary support structure 1250 fully raised, such that the actuator housing 1270 fully expands the provided telescoping cowling.

In a second configuration, shown in FIG. 19, the mattress platform 1240 is supported relative to the surface 150 by the primary support structure 1250 fully lowered, such that the actuator housing 1270 fully contracts.

The mattress support 1230 shown may function as a retrofit, but it may also be provided with the crib 1200 as shown. Such an assembly or kit would then include a crib enclosure 1210 defined by a plurality of walls 1220. A first support structure (not shown) would then be provided for supporting the walls 1220 relative to a surface. The mattress platform 1240 would then be translatable vertically relative to the plurality of walls 1220 and within the crib enclosure 1210.

A second support structure, typically the primary support structure 1250 discussed above, would then be fixed to the mattress platform 1240 for supporting the mattress support relative to the surface 150. An actuator 1270 would be provided for vertically translating the mattress platform 1240 relative to the second support 1250.

The aftermarket, or retrofit, version of the bed lift may consist of a standard scissor lift, a bed platform, and an enclosure designed to prevent users and children from accessing the mechanism or encountering pinch points.

The standard scissor lift may be activated via a linear actuator and have, e.g., 3 positions. The lowest position may then be the device minimum position and is set to be as low as possible to fit under standard cribs. The second position may then be user set and may be used for the “lower” sleeping position. Such a position may be approximately 26 inches from the height of the top rail of the crib. The highest position may similarly be user defined, or may be a maximum extension of the device, and may bring the platform up by approximately 9-11 inches to approximately 15 inches from the top rail of the crib.

The enclosure may be bounded on the top and bottom by a rigid panel. It may consist of a telescoping cowling which can adjust from the minimum height to the maximum height without exposing any internal mechanisms or pinch points. This may be achieved with a single or double nested interlocking design. Double nesting is shown below to accommodate the largest range of crib heights.

The bed platform can consist of two or more rigid panels designed to assemble into a full size platform for a standard sized crib (approx. 52” x 28”). When cut in half, this platform can be shipped in a footprint that fits the main device footprint.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A crib comprising:
a plurality of walls at least partially defining a crib enclosure;
a primary support structure for supporting the plurality of walls relative to a surface;
a fixation structure fixed relative to the plurality of walls;
a mattress support translatable vertically relative to the fixation structure and within the crib enclosure;
a secondary support structure fixed to the mattress support for supporting the mattress support relative to the surface; and
an actuator for vertically translating the mattress support relative to the fixation structure.

2. The crib of claim 1 wherein the crib has:
a first configuration wherein the crib and mattress support are supported relative to the surface by the primary support structure and not the secondary support structure, and
a second configuration wherein the crib and mattress support are supported relative to the surface by the secondary support structure and not the primary support structure.

3. The crib of claim 2 wherein the crib has a third configuration in which both the primary support structure and the secondary support structure are simultaneously in contact with the surface.

4. The crib of claim 2 wherein a height of the mattress support is continuously adjustable within its range of motion, such that the mattress support can be set to a plurality of heights within the first configuration.

5. The crib of claim 2 wherein the secondary support structure further comprises a plurality of wheels or casters and wherein, in the second configuration, the crib and mattress support are supported relative to the surface by the wheels or casters such that in the second configuration, the crib is mobile.

6. The crib of claim 1 wherein the actuator comprises at least one of a scissor jack, a screw jack, a linear actuator, a motorized actuator, and a manually operated mechanical actuator.

7. The crib of claim 6 wherein the actuator is configured to set the mattress support to one of a plurality of predetermined heights.

8. The crib of claim 1 further comprising a sensor for determining if an obstruction is in a path of the mattress support wherein a controller is configured to prevent movement of the mattress support upon detection of an obstruction by the sensor.

9. The crib of claim 1 further comprising a weight sensor for determining a weight of an object placed on the mattress support, and wherein the mattress support has a primary maximum weight at which actuation of the actuator is safe, and wherein a determination that the weight of the object is greater than the primary maximum weight prevents movement of the actuator.

10. The crib of claim 9 wherein the mattress support has a secondary maximum weight at which use of the mattress support is safe, wherein the secondary maximum weight is higher than the primary maximum weight.

11. The crib of claim 1 further comprising a sound generator for generating white noise, wherein the sound generator obscures any noise generated by a motor associated with the actuator.

12. The crib of claim 1 further comprising a sensor for detecting heart rate variability data associated with a child located within the crib.

13. The crib of claim 1 wherein the primary support structure comprises a first plurality of legs for supporting the plurality of walls, and wherein the secondary support structure comprises a second plurality of legs fixed to the mattress support.

14. The crib of claim 1 wherein the fixation structure is a platform or shelf fixed to at least one of the plurality of walls and to which the actuator is mounted.

15. The crib of claim 1 wherein the fixation structure is a mounting structure or location integrated into at least one of the plurality of walls for mounting the actuator.

16. A mattress support for use in a crib comprising:
a mattress platform sized for supporting a crib mattress within a crib enclosure;
a primary support structure for supporting the mattress platform relative to a surface; and
an actuator for vertically translating the mattress platform relative to a foot of the primary support structure.

17. The mattress support of claim 16 wherein the actuator is located within the primary support structure, such that the primary support structure expands or contracts in order to adjust a height of the mattress platform.

18. The mattress support of claim 17 further comprising a primary support structure housing for fully enclosing the actuator.

19. The mattress support of claim 16, wherein the mattress platform comprises a plurality of independent panels, wherein a footprint of the primary support structure corresponds substantially to a size of each of the plurality of independent panels.

20. A crib comprising:
a plurality of walls at least partially defining a crib enclosure;
a first support structure for supporting the plurality of walls relative to a surface;
a mattress platform translatable vertically relative to the plurality of walls and within the crib enclosure;
a second support structure fixed to the mattress platform for supporting the mattress support relative to the surface; and
an actuator for vertically translating the mattress platform relative to the second support.
